(19) United States
(12) Patent Application Publication
(10) Pub. No.: US 2026/0283659 A1
(43) Pub. Date: Sep. 24, 2026

(54) ORTHOPEDIC INTRAMEDULLARY NAIL AND BONE PLATE SYSTEM

(71) Applicants: Smith & Nephew, Inc., Memphis, TN (US); Smith & Nephew Orthopaedics AG, Zug (CH)

(72) Inventors: Charles R. Bennett, JR., Matthews, NC (US); Nicholas S. Ritchey, Collierville, TN (US); Cesar Zabdiel Gutierrez Mendoza, Olive Branch, MS (US); Nathaniel K. Grusin, Germantown, TN (US); Gabriel E. Rapalo, Arlington, TN (US); Jordan Rakes, Philadelphia, PA (US)

(73) Assignees: Smith & Nephew, Inc., Memphis, TN (US); Smith & Nephew Orthopaedics AG, Zug (CH)

(21) Appl. No.: 19/671,474

(22) Filed: May 8, 2026

Related U.S. Application Data
(63) Continuation-in-part of application No. PCT/US2025/032044, filed on Jun. 3, 2025.
(60) Provisional application No. 63/658,083, filed on Jun. 10, 2024.

(51) Int. Cl. A61B 17/72; A61B 17/80; A61B 17/90

(52) U.S. Cl. CPC A61B 17/725; A61B 17/8042; A61B 17/8052; A61B 17/8061; A61B 17/808; A61B 17/90

(57) ABSTRACT

An intramedullary (“IM”) nail-bone plate system, and corresponding instrumentation and method of use, for fractured fixation of a patient's bone is disclosed. The IM nail-bone plate system may include an IM nail including a plurality of locking screw openings, a bone plate including one or more elongated, non-locking screw openings, and a bone screw insertable into the elongated, non-locking screw opening formed in the bone plate and into the locking screw opening formed in the IM nail. In some examples, the locking screw opening formed in the IM nail may be configured as an elongated slot so that the bone screw is inserted through the elongated, non-locking screw opening formed in the bone plate and into the elongated slot formed in the IM nail. Thus arranged, independent positioning of the IM nail, the bone plate, or a combination thereof, is enabled.

### CROSS-REFERENCE TO RELATED APPLICATIONS

This application is a continuation-in-part of International PCT Application No. PCT/US2025/032044, filed Jun. 3, 2025, which application is a non-provisional of, and claims the benefit of the filing date of, U.S. provisional patent application No. 63/658,083, filed Jun. 10, 2024, entitled “Orthopedic Intramedullary Nail and Bone Plate System” the entirety of each application is incorporated by reference herein.

### FIELD OF THE DISCLOSURE

The present disclosure is directed to orthopedic implants, and more specifically to an intramedullary (“IM”) nail and bone plate system.

### BACKGROUND

Orthopedic implants may be used, for example, to stabilize an injury, to support a bone fracture, to fuse a joint, and/or to correct a deformity. Orthopedic implants may be attached permanently or temporarily, and may be attached to the bone at various locations, including implanted within an intramedullary canal or other cavity of the bone, implanted beneath soft tissue and attached to an exterior surface of the bone, or disposed externally and attached by fasteners such as screws, pins, and/or wires. Some orthopedic implants allow the position and/or orientation of two or more bones, bone pieces, or bone fragments (terms used interchangeably herein without the intent to limit or distinguish) to be adjusted relative to one another. Orthopedic implants are generally machined or molded from isotropic materials, such as metals including, for example, titanium, titanium alloys, stainless steel, cobalt-chromium alloys, tantalum, etc.

An intramedullary (“IM”) nail is one type of orthopedic implant. The primary function of the IM nail is to stabilize the fractured bone fragments, and thereby enable load transfer across the fracture site while maintaining anatomical alignment of the bone. Another type of orthopedic implant is a bone plate. Currently, there are a large number of different commercially available IM nails and bone plates in the marketplace.

One disadvantage of IM nails is that during fracture fixation there may be fractured fragments, which cannot be captured using the IM nail and screw offerings alone. That is, for example, bone fractures such as, for example, femoral fractures, humeral fractures, or the like, may include a two-part fracture, where the head or a portion of the head may be broken from the shaft of the patient's bone. In addition, the head fracture may include two or three fragments that separate from the shaft. Moreover, poor bone purchase resulting from, for example, osteoporosis, may further complicate the surgical procedure.

One known solution for treating such fractures may involve the use of additional cables, sutures, and/or additional bone plates to capture the fractured bone fragments. For example, an IM nail may be inserted into the intramedullary canal of the patient's bone and one or more screws may be inserted into the head of the bone. In addition, fractured fragments may be secured together by an elongated bone plate that is secured to the shaft of the patient's bone.

However, there remains a need to provide improved orthopedic fixation of a patient's fractured bone. The present disclosure satisfies this need and provides other benefits and advantages in a novel and unobvious manner.

### BRIEF SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

An intramedullary (“IM”) nail-bone plate combo system for fractured fixation of a patient's bone is disclosed. In some examples, the IM nail-bone plate combo system includes an IM nail, a bone plate, and one or more bone screws. The IM nail is arranged and configured to be inserted into an intramedullary canal of a patient's bone. The IM nail includes a plurality of locking screw openings. The bone plate is arranged and configured to be positioned against the patient's bone. The bone plate includes a plurality of screw openings and one or more elongated, non-locking screw openings. In use, at least one bone screw is arranged and configured to be inserted into the one or more elongated, non-locking screw opening formed in the bone plate and into one of the plurality of locking screw openings formed in the IM nail.

In any preceding or subsequent example, at least one of the plurality of locking screw openings formed in the IM nail is configured as an elongated slot so that the at least one bone screw is configured to be inserted through one of the elongated, non-locking screw openings formed in the bone plate and into the elongated slot formed in the IM nail.

In any preceding or subsequent example, the elongated slot formed in the IM nail and the elongated, non-locking screw opening formed in the bone plate enable independent positioning of the IM nail, the bone plate, or a combination thereof.

In any preceding or subsequent example, the plurality of screw openings formed in the bone plate include a plurality of locking screw openings and a plurality of non-locking variable angled screw openings.

In any preceding or subsequent example, the plurality of screw openings formed in the bone plate include one or more variable angled nail screw openings arranged and configured to receive a larger diameter nail screw.

In any preceding or subsequent example, the one or more variable angled nail screw openings include a plurality of concavely indented, inwardly protruding fins defining a non-threaded opening for cooperating with threads formed on a nail screw, wherein each of the fins include a terminal tip, a base, and a tapered surface such that the tip includes a first thickness at the tip arranged and configured to deform, the base includes a second thickness greater than the first thickness, the second thickness sufficient to prevent deformation.

In any preceding or subsequent example, the nail screw includes threads having a pitch configured to be inserted into a patient's bone.

In any preceding or subsequent example, the nail screw includes a continuous thread extending an entire length thereof.

In any preceding or subsequent example, the first thickness is approximately 8/1000 of an inch.

In any preceding or subsequent example, the second thickness is equal to approximately a pitch of the threads formed on the nail screw.

A method of repairing a fractured bone is also disclosed. In some examples, the method includes inserting an intramedullary (“IM”) nail into an intramedullary canal of the fractured bone, the IM nail including one or more locking openings. Positioning a bone plate against the fractured bone B, the bone plate including one or more elongated, non-threaded linking openings. Coupling a targeting jig assembly to the IM nail, the targeting jig assembly including a targeter including one or more openings formed therein for targeting the one or more elongated, non-threaded linking openings formed in the bone plate. Inserting one or more guide sleeves into the one or more openings formed in the targeter. Inserting one or more provisional fixation pins through the one or more guide sleeves, through the one or more elongated, non-threaded linking openings formed in the bone plate, and into the or more locking openings formed in the IM nail. Optionally, adjusting a position of the bone plate relative to the IM nail.

In any preceding or subsequent example, the method further includes removing the one or more guide sleeves, and rotating the targeter relative to the IM nail to provide increased access space to the surgeon.

In any preceding or subsequent example, the method further includes inserting one or more bone screw through one or more openings formed in the bone plate, the one or more bone screws avoiding the IM nail.

In any preceding or subsequent example, the method further includes rotating the targeter relative to the IM nail to its original position, re-inserting the one or more guide sleeves, removing the one or more provisional fixation pins, and inserting one or more bone screw through the one or more guide sleeves, through the one or more elongated, non-threaded linking openings formed in the bone plate, and into the or more locking openings formed in the IM nail.

In any preceding or subsequent example, the targeting jig assembly further includes a rod coupled to the IM nail and a guide handle coupled to the rod and the targeter, wherein the guide handle is rotatably coupled to the rod between a first position, wherein the one or more openings formed in the targeter are aligned with the one or more elongated, non-threaded linking openings formed in the bone plate, and a second position, wherein the one or more openings formed in the targeter are not aligned with the one or more elongated, non-threaded linking openings formed in the bone plate.

An orthopedic implant is also disclosed. In some examples, the orthopedic implant includes a first surface, a second surface, and a plurality of variable angled openings extending from the first surface to the second surface. Each of the plurality of variable angled opening including a plurality of concavely indented inwardly protruding fins defining a non-threaded opening for cooperating with threads formed on a screw. In some examples, each of the fins include a terminal tip, a base, and a tapered surface such that the tip includes a first thickness at the tip arranged and configured to deform, the base includes a second thickness greater than the first thickness, the second thickness sufficient to prevent deformation.

In any preceding or subsequent example, each of the plurality of variable angled opening is arranged and configured to receive a nail screw including threads having a pitch configured to be inserted into a patient's bone.

In any preceding or subsequent example, the nail screw includes a continuous thread extending an entire length thereof.

In any preceding or subsequent example, the first thickness is approximately 8/1000 of an inch.

In any preceding or subsequent example, the second thickness is equal to approximately a pitch of the threads formed on the nail screw.

Examples of the present disclosure provide numerous advantages. For example, in accordance with one or more features of the present disclosure, the IM nail-bone plate combo system enable selective and independent positioning of the IM nail and/or bone plate thereby providing surgeons with increased flexibility in positioning the bone plate relative to the IM nail.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 is a perspective view of an example of IM nail-bone plate combo system including associated instrumentation in accordance with various features of the present disclosure, the IM nail-bone plate combo system shown implanted within a patient's bone;

FIG. 2 is a perspective view of the IM nail-bone plate combo system shown in FIG. 1;

FIG. 3 is a perspective view of an example IM nail, which may be used in the IM nail-bone plate combo system shown in FIG. 2;

FIG. 4A is a top view of an example bone plate, which may be used in the IM nail-bone plate combo system shown in FIG. 2;

FIG. 4B illustrates an alternate top view of the bone plate shown in FIG. 4A;

FIG. 5A is an alternate perspective view of the IM nail-bone plate combo system shown in FIG. 2;

FIG. 5B is a detailed, perspective view of the IM nail-bone plate combo system shown in FIG. 5A;

FIG. 5C is an alternate detailed, perspective view of the IM nail-bone plate combo system shown in FIG. 5A, FIG. 5C illustrating a bone screw positioned in an empty or opened space defined by a corner cut formed on the bone plate, the bone screw engaging the IM nail while avoiding the bone plate;

FIGS. 5D and 5E illustrate perspective views of the IM nail-bone plate combo system shown in FIG. 5A, FIGS. 5D and 5E illustrating an example of a range of adjustment between the bone plate and IM nail in accordance with one or more features of the present disclosure;

FIG. 6A is a side view of an example bone screw, which may be used in the IM nail-bone plate combo system shown in FIG. 2;

FIG. 6B is a side view of an example nail screw in accordance with one or more features of the present disclosure, which may be used in the IM nail-bone plate combo system shown in FIG. 2;

FIG. 7A is a cross-sectional, perspective view of an example variable angled opening in accordance with one or more features of the present disclosure, which may be incorporated into an orthopedic implant such as, for example, the bone plate shown in FIG. 4A;

FIG. 7B is a cross-sectional view of the variable angled opening shown in FIG. 7A;

FIG. 7C is a cross-sectional view of the nail screw shown in FIG. 6B inserted into the variable angled opening shown in FIG. 7A;

FIG. 8A is a perspective view of an example instrumentation such as, for example, a targeting jig assembly, in accordance with one or more features of the present disclosure, the targeting jig assembly coupled to the IM nail for targeting openings formed in the IM nail-bone plate combo system shown in FIG. 1;

FIG. 8B is an alternate perspective view of the targeting jig assembly shown in FIG. 8A, the targeting jig assembly including one or more guide sleeves;

FIG. 8C is an alternate perspective view of the targeting jig assembly shown in FIG. 8B, the targeting jig assembly including one or more provisional fixation pins in accordance with one or more features of the present disclosure;

FIG. 8D is an alternate perspective view of the targeting jig assembly shown in FIG. 8C;

FIG. 8E is an alternate perspective view of the targeting jig assembly shown in FIG. 8D, the targeting jig assembly illustrated in a swiveled position;

FIG. 9 is a perspective view of an example provisional fixation pin in accordance with one or more features of the present disclosure, which may be used in the IM nail-bone plate combo system shown in FIG. 2;

FIGS. 10A-10F illustrate alternate examples of an IM nail-bone plate combo system including multiple bone plates;

FIGS. 11A and 11B illustrate an alternate example of an IM nail-bone plate combo system including a set screw; and

FIG. 12 illustrates an alternate example of an IM nail-bone plate combo system including an internal set screw positioned within the IM nail.

It should be understood that the drawings are not necessarily to scale and that the disclosed examples are sometimes illustrated diagrammatically and in partial views. In certain instances, details which are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

Various features or the like of an orthopedic intramedullary (“IM”) nail and bone plate combo system and/or corresponding instrumentation and/or method of use will now be described more fully hereinafter with reference to the accompanying drawings, in which one or more features of the orthopedic IM nail and bone plate combo system, instrumentation, and/or method of use will be shown and described. It should be appreciated that the various features may be used independently of, or in combination, with each other. It will be appreciated that the orthopedic IM nail and bone plate combo system, instrumentation, and/or method of use as disclosed herein may be embodied in many different forms and should not be construed as being limited to the specific examples set forth herein. Rather, these examples are provided so that this disclosure will convey certain features to those skilled in the art.

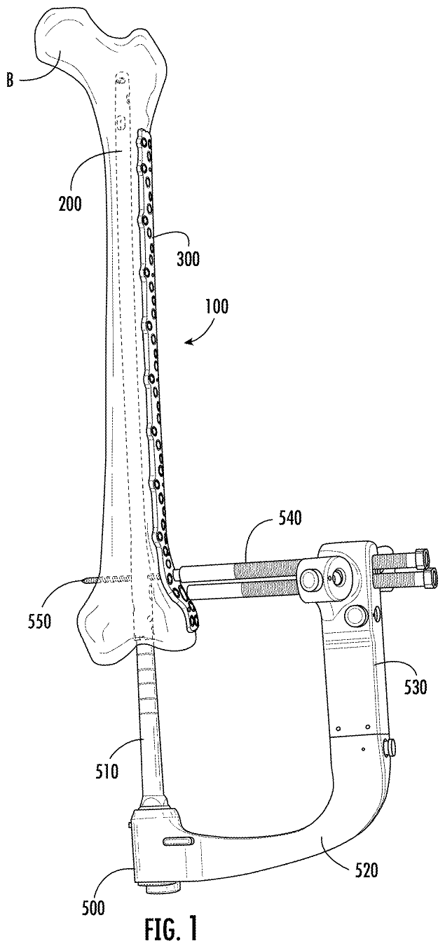

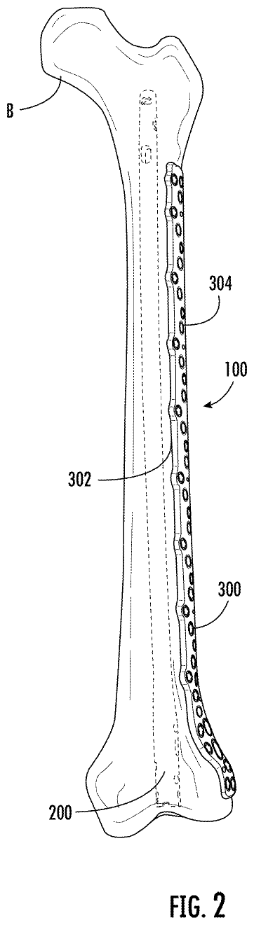

With reference to FIGS. 1, 2, 5A, and 5B, in accordance with one or more features of the present disclosure, an IM nail-bone plate system 100 is disclosed. As illustrated, the IM nail-bone plate system 100 includes an IM nail 200, a bone plate 300, and one or more bone screw 400 with at least one bone screw being inserted through a linking opening 330 formed in the bone plate 300 for engaging the IM nail 200. As will be described, additional bone screw 400 may be used to fasten the bone plate 300 to the patient's bone B.

While a specific IM nail 200 and bone plate 300 will be described and illustrated herein, it should be appreciated that any configuration of IM nail now known or hereafter developed may be utilized. In addition, any configuration of bone plate now known or hereafter developed may be utilized. As will be appreciated by one of ordinary skill in the art, the configuration of IM nail and bone plate may be party determined by the area of the patient's body being fixated such as, for example, the femur, tibia, humerus, etc. As such, the present disclosure should not be limited to any particular configuration of IM nail and/or bone plate except as specifically claimed.

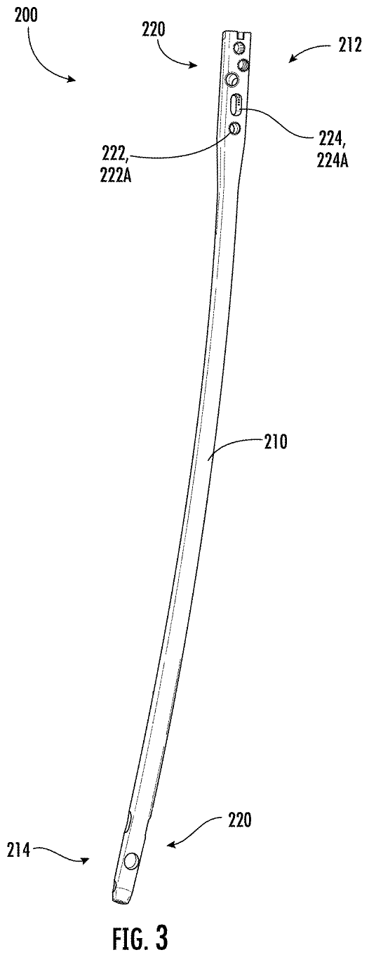

Referring to FIG. 3, an example of an IM nail 200, which may be used in the IM nail-bone plate system 100 is illustrated. As illustrated, and as will be appreciated by one of ordinary skill in the art, the IM nail 200 is arranged and configured to be inserted into the intramedullary canal of a patient's bone B. For example, as illustrated, the IM nail 200 may be arranged and configured to be implanted into the intramedullary canal of a patient's femur, although this is but one configuration. As such, the IM nail 200 may also be referred to as a femoral nail. Generally speaking, the IM nail 200 includes a body 210 such as, for example, a cannulated body. The body 210 includes a first or proximal end portion 212 and a second or distal end portion 214. The first or proximal end portion 212 and the second or distal end portion 214 including a plurality of screw openings, holes, slots, etc. 220 (terms used interchangeably herein without the intent to limit) arranged and configured to receive a bone fastener, screw, etc. (terms used interchangeably herein without the intent to limit or distinguish). In some examples, as will be described in greater detail below, the screw openings 220 may be in the form of a locking screw (or fastener) opening such as, for example, include threads for engaging the bone screw, include a locking ridge for engaging the bone screw, or the like. Alternatively, the screw openings 220 may be in the form of a non-locking or variable angled screw (or fastener) opening for enabling variable-angled positioning of the bone screw, or have any other configuration now known or hereafter developed. For reasons that will become apparent below, by providing a locking screw opening, the IM nail 200 facilitates locking of the bone screw inserted therein.

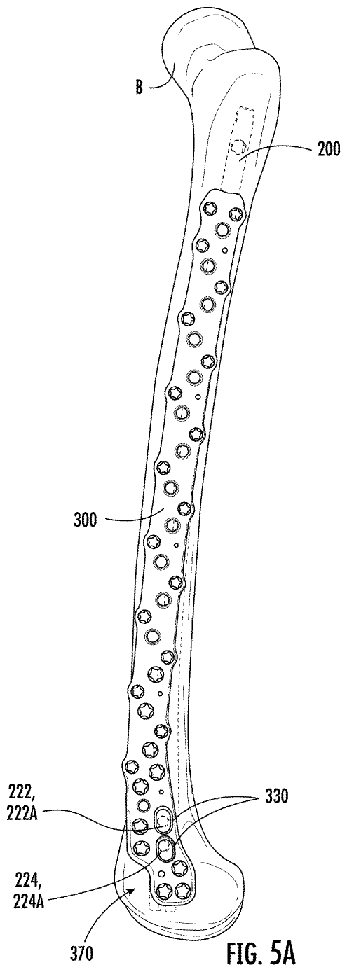

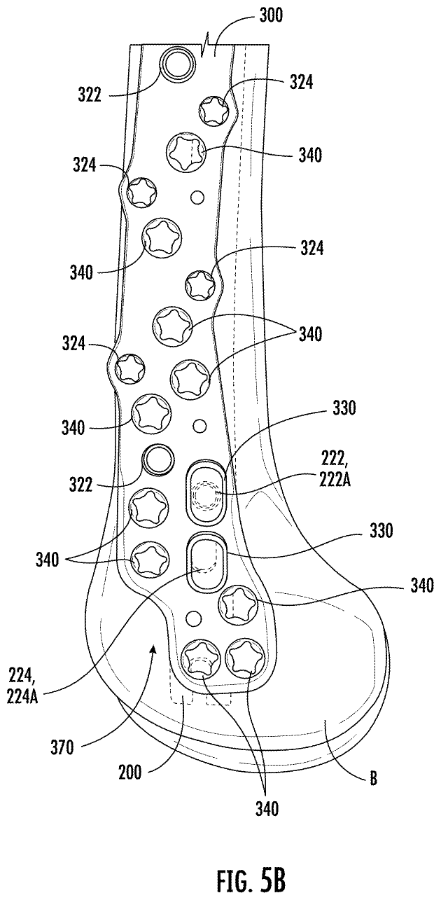

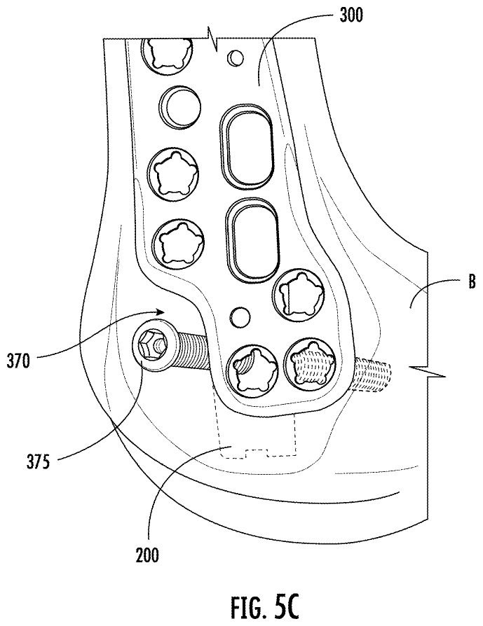

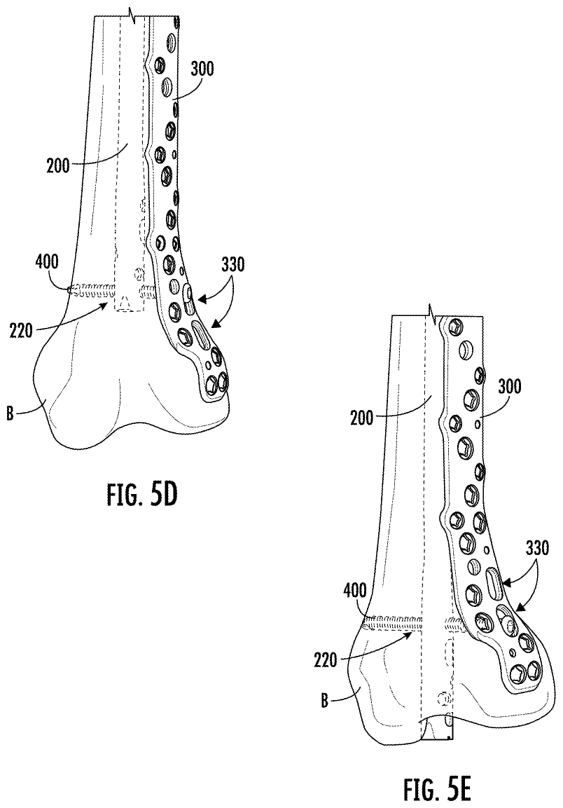

With additional reference to FIGS. 5A and 5B, in accordance with one or more features of the present disclosure, the first or proximal end portion 212 includes one or more locking screw openings 222. In addition, the first or proximal end portion 212 may include one or more elongated slots 224. For example, as illustrated, the first or proximal end portion 212 include a first locking screw opening 222A and a first locking elongated slot 224A.

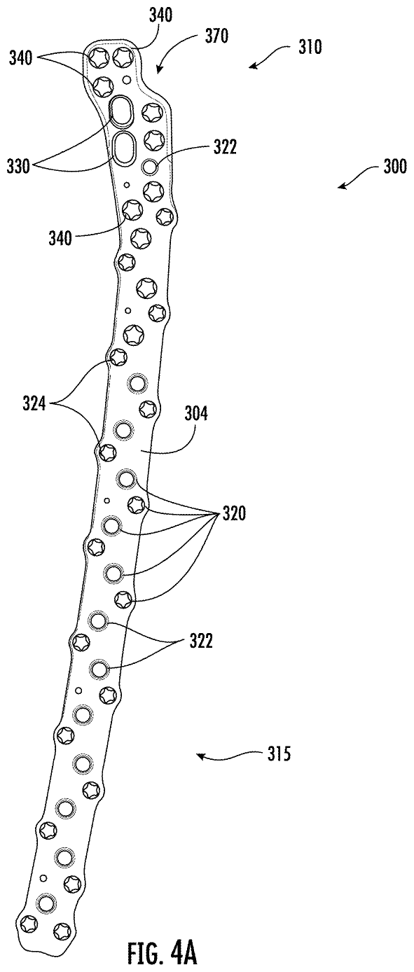

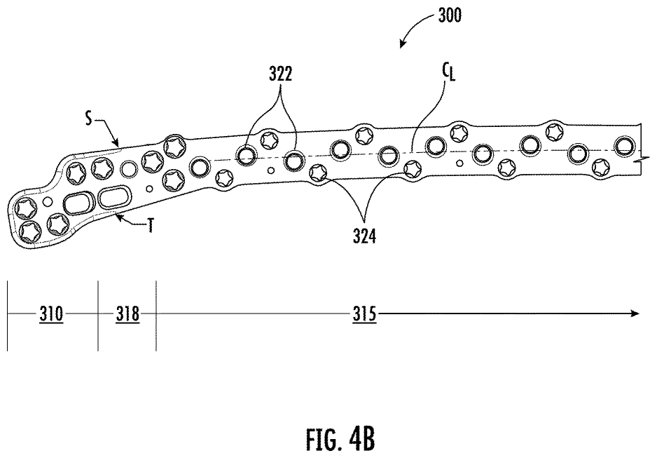

Referring to FIG. 4A, an example of a bone plate 300, which may be used in the IM nail-bone plate system 100 is illustrated. As illustrated, and as will be appreciated by one of ordinary skill in the art, the bone plate 300 is arranged and configured to be disposed on a surface of the patient's bone B.

As will be described herein, the bone plate 300 may include one or more features for enabling increased flexibility for coupling the bone plate to the patient's bone B adjacent to the IM nail 200. In addition, the bone plate 300 includes one or more features for coupling the bone plate 300 to the IM nail 200 via one or more bone screws 400. As such, as will be described herein, the present disclosure discloses one or more features that may be used in combination or singularly, these features are designed and configured to provide increased flexibility in enabling a surgeon to position and secure the bone plate 300 to patient's bone B adjacent to the IM nail 200 and/or to secure the bone plate 300 to the IM nail 200.

As will be described herein, the bone plate 300 may have various shapes and/or configurations. It should be appreciated that the bone plate 300 may be provided in any suitable shape and/or configuration, which, as will be appreciated by one of ordinary skill in the art, may be dependent on the location and type of patient's bone being fixed. For example, the bone plate 300 may be in the form of a proximal femur plate. That is, the bone plate 300 may be arranged and configured for positioning adjacent to the proximal femur of the patient, although this is but one configuration. For example, the bone plate 300 may be arranged and configured to span, contact, etc. a distal femur, a distal tibia, a proximal tibia, a proximal humerus, a distal humerus, etc.

As illustrated, the bone plate 300 includes an underside, lower, or bone facing surface 302 (terms used interchangeably herein without the intent to limit) and an upper surface 304. In addition, the bone plate 300 may include a head portion 310 and a shaft portion 315. Moreover, the bone plate 300 includes a plurality of openings 320 formed therein for receiving a plurality of bone screws 400 for coupling the bone plate 300 to the patient's bone B.

As illustrated, in some examples, the head portion 310 may include a corner cut 370. That is, a portion of the head portion 310 may be removed thereby defining an empty or opened space or void. As illustrated, the corner cut 370 may be defined by inwardly projecting, tapering, contouring, etc. a side surface or periphery of the bone plate 300. Thus arranged, as illustrated, a portion of the bone plate 300 such as, for example, the head portion 310, may include a reduced width thereby defining an empty or opened space or void, which, in use, as best illustrated in FIG. 5C, a surgeon may position a bone screw 375 in the empty or opened space or void defined by the corner cut 370. As such, the bone screw 375 can engage one of the screw openings 220 formed in the IM nail 200 while avoiding the bone plate 300 in situations where, for example, the surgeon does not want to unitize or couple the bone plate 300 to the IM nail 200. In addition, it has been discovered that providing the corner cut 370 facilitates reducing patient irritation.

As will be described herein, the openings 320 may be in the form of a locking screw (or fastener) opening 322 or a variable angled opening or variable angled fastener (or screw) opening 324 (terms used interchangeably herein without the intent to limit). Preferably in some examples, as illustrated, the bone plate 300 includes a plurality of locking screw openings 322 and a plurality of variable angled openings 324 to provided increased flexibility during a surgical procedure for surgeons to position screws to be implanted within the patient's bone while avoiding the IM nail 200.

As will be appreciated by one of ordinary skill in the art, locking screw openings 322 may include a plurality of threads formed on an inner surface thereof for mating with threads formed on an outer surface of a head portion of the bone screw. Thus arranged, the bone screw may be said to be locked to the bone plate 300 via the threads on the head of the bone screw and the threads of the locking screw opening 322. That is, as will be appreciated by one of ordinary skill in the art, the bone screw is threaded through one of the locking screw openings 322 formed in the bone plate 300 and into the patient's bone. The bone screw is secured to the bone plate 300 via threads formed on the head portion of the bone screw that cooperate with the threaded locking screw opening 322 formed in the bone plate 300. This secures the bone plate 300 with respect to the patient's bone and provides rigid fixation between the bone plate 300 and the bone screws. That is, because the head portion of the bone screw interdigitates with the threads formed in the locking screw openings 322 of the bone plate 300, the bone plate 300 and the bone screws form a stable system or construct, and the stability of the fracture can be dependent on or aided by the stiffness of the construct. Locking a bone screw into the bone plate 300 can achieve angular and axial stability and eliminate the possibility for the bone screw to toggle, slide, or be dislodged, reducing the risk of postoperative loss of reduction.

As previously mentioned, the bone plate 300 may also include a plurality of variable angled openings 324 formed therein for receiving a non-locking or variable angled (e.g., polyaxial) bone screw. In use, the variable angled openings 324 are arranged and configured to enable the bone screw inserted therein to achieve a greater range of insertion angles as compared to, for example, a conventional locking screw that is threadably coupled to the bone plate 300. For example, the angular position of the bone screw may be rotated through a range of approximately ±15 degrees, although the range of allowable polyaxial rotation can vary, including greater and less than fifteen degrees. In use, the variable angled openings 324 may be provided in any suitable manner, configuration, etc. now known or hereafter developed for enabling polyaxial positioning or angling of the bone screw relative to the bone plate 300.

As generally illustrated, in some examples, the variable angled openings 324 may include fins or projections that extend radially inward from an inner surface of the variable angled openings 324 and into an interior region of the variable angled openings 324, and which are configured to engage or cooperate with the head portion of the bone screw. In use, the fins engage, interdigitate, etc. with the threads formed on the head portion of the bone screw in order to secure the bone screw at a desired position and at a desired angular orientation within the variable angled opening 324.

That is, the finned, variable angled openings 324 may include an inner surface and a plurality of inwardly protruding fins that extend toward a central axis of the finned, variable angled openings 324. Each fin including a base, a terminal end or tip, and side surfaces. In use, the entire inner surface of the finned variable angled openings 324 can be devoid of any threads. The bases can extend from the inner surface of the finned variable angled openings 324. In some examples, the fins are integrally connected to, and protruding from, the inner surface of the finned, variable angled openings 324. Thus arranged, the finned, variable angled openings 324 may be seen to have a jagged circumference formed by protruding fins.

As shown, in some examples, as the fins extend toward the central axis of the finned, variable angled openings 324, the fins can taper to form inwardly tapered side surfaces. Alternatively, the side surfaces of the fins may taper outwardly or may be parallel with each other. The terminal ends or tips can have any shape suitable for engaging the head portion of the bone screw. For example, the terminal ends or tips may include concave portions, which are smooth and non-threaded for interdigitating with the threads of the bone screw. Alternatively, the terminal ends or tips can be rounded, pointed, square, rectangular, or any other appropriate configuration.

In use, the fins may be provided as a series of concavely indented, inwardly protruding fins that are adapted to secure a head of a bone screw in place at varying angles (e.g., fins engage the threads or other surfaces formed on the head portion of the bone screw).

By providing a non-threaded inner surface, the bone screw can be inserted into the finned, variable angled openings 324 at a desired insertion angle (e.g., an angle between a longitudinal axis of the bone screw and the central axis of the finned, variable angled openings 324). The central axis and the longitudinal axis can be co-linear so that the insertion angle is zero, or the central axis and the longitudinal axis can be non-co-linear with an insertion angle of up to about +/−15 degrees. Varying the insertion angle is possible because there are no threads in the finned, variable angled openings 324 to interfere with the desired insertion angle. In use, the fins may be configured to slightly bend or deform in order to secure the bone screw in place in the finned, variable angled openings 324.

In some examples, the locking screw openings 322 may be larger than the variable angled openings 324 (e.g., the plurality of locking screw openings 322 may include a first diameter and the plurality of variable angled openings 324 may include a second diameter, the first diameter being larger than the second diameter). For example, the locking screw openings 322 may be sized and configured to receive, for example, 4.5 mm locking screws. The variable angled openings 324 may be sized and configured to receive, for example, 3.5 mm bone screws. Alternatively, in some examples, the locking screw openings 322 and the variable angled openings 324 may be the same size.

In addition, and/or alternatively, as best illustrated in FIG. 4B, the variable angled openings 324 may be positioned, for example, along a periphery of the shaft portion 315 while the locking screw openings 322 may be more centrally located. That is, the locking screw openings 322 may be more centrally located as compared to the variable angled openings 324. Thus, the variable angled openings 324 may be positioned along and/or adjacent to an outer periphery or surface of the bone plate 300. For example, the shaft portion 315 may include a central longitudinal axis C_{L}, the locking screw openings 322 may be positioned substantially along the central longitudinal axis C_{L} of the shaft portion 315 while the variable angled openings 324 may be positioned along and/or adjacent to an outer periphery or surface of the shaft portion 315. That is, the locking screw openings 322 may be positioned more interior, closer to the central longitudinal axis C_{L} of the shaft portion 315 relative to the variable angled openings 324, which may be positioned closer to the outer periphery or perimeter surface of the shaft portion 315. Thus arranged, by positioning the variable angled openings 324 along and/or adjacent to the outer periphery, the bone plate 300 is better able to position the variable angled bone fastener to avoid the IM nail 200 (e.g., the surgeon is better able to position and insert one or more bone fasteners through the variable angled openings 324 formed in the bone plate 300 while avoiding the IM nail 200).

In addition, and/or alternatively, the bone plate 300 may have any suitable thickness now known or hereafter developed. In some examples, the bone plate 300 may include a thickness arranged and configured to enable the bone plate 300 to facilitate contouring of the bone plate 300 to the patient's anatomy in at least sections thereof. For example, the bone plate may include a constant thickness of less than 3.5 mm.

Alternatively, in some examples, the bone plate 300 may include a varying thickness across sections or portions of the bone plate 300. For example, as illustrated in FIG. 4B, the bone plate 300 may include a first thickness in the head portion 310 of the bone plate 300. In one exemplary example, the head portion 310 may include a thickness (e.g., first thickness) of approximately 1.5 mm to 2.0 mm, and preferably 1.7 mm.

In addition, and/or alternatively, as illustrated in FIG. 4B, the bone plate 300 may include a second thickness in the shaft portion 315 of the bone plate 300. The shaft portion 315 may include a thickness (e.g., second thickness) of approximately 3.0 mm to 3.3 mm, and preferably 3.2 mm.

In addition, and/or alternatively, as illustrated in FIG. 4B, the bone plate 300 may include an optional third thickness at the joint or transition 318 between the head portion 310 and the shaft portion 315. The transition 318 may include a thickness (e.g., third thickness) of approximately 3.0 mm to 3.2 mm, and preferably 3.1 mm.

In addition, and/or alternatively, while the bone plate 300 may be manufactured from any suitable material now know or hereafter developed, the bone plate 300 may preferably be manufactured from titanium to further facilitate contouring of the bone plate 300.

Although these are but some examples, and the bone plate 300 may include different thicknesses and/or the first, second, and optional third thicknesses may be used in combination or separately from the others.

Additional information on the operation and configuration of the fins can be found in U.S. patent application Ser. No. 15/706,877, with an earliest filing date of Jul. 25, 2005, now U.S. Pat. No. 10,092,337 entitled “Systems and Methods for Using Polyaxial Plates”; U.S. patent application Ser. No. 13/524,506, filed on Jun. 15, 2012, entitled “Variable Angle Locking Implant”, and International PCT Patent Application No. PCT/US20/35729, filed on Jun. 2, 2020, entitled “Orthopedic Implant with Improved Variable Angle Locking Mechanism”, the entire contents of which are hereby incorporated by reference.

Additional information on the operation and configuration of exemplary bone plates can be found in U.S. patent application Ser. No. 17/772,319, filed on Apr. 27, 2022, entitled “Periprosthetic Bone Plate Systems” and U.S. patent application Ser. No. 17/921,721, filed Oct. 27, 2022, entitled “Periprosthetic Bone Plate.”

As best illustrated in FIGS. 5A and 5B, in accordance with one feature of the present disclosure, the bone plate 300 includes one or more linking openings 330 formed therein. As illustrated, the one or more linking openings 330 may be configured as elongated openings or slots. In addition, as illustrated, the one or more linking openings 330 are arranged and configured as non-locking screw openings (e.g., variable angled openings). In use, the one or more linking openings 330 are arranged and configured to enable a screw to pass through the bone plate 300 and into engagement with one of the locking screw openings 222 or one of the locking elongated slots 224 formed in the IM nail 200 such as, for example, the first locking screw opening 222A and/or the first locking elongated slot 224A. Thus arranged, the screws inserted through the one or more linking openings 330 are arranged and configured to link, couple, engage, or the like, the bone plate 300 to the IM nail 200. As such, in use, the screw is non-threadable coupled to the bone plate 300 (e.g., the screw is secured or locked to the IM nail 200 but not the bone plate 300 (i.e., the screw passes through the elongated, non-threaded linking opening 330 formed in the bone plate 300)). Thus, the bone plate 300 provides rotational stability relative to the IM nail 200 (e.g., linking the bone plate 300 to the IM nail 200 rotational stabilizes the bone plate 300 relative to the IM nail 200).

Thus arranged, by providing one or more linking openings 330 in the form of non-threaded, elongated slots or openings in the bone plate 300, surgeons are provided with increased flexibility in independently positioning the IM nail 200 and/or the bone plate 300 (e.g., elongated slots formed in the bone plate 300 facilitate optimized positioning and/or alignment of the bone plate 300 relative to the IM nail 200). That is, surgeons are provided with increased flexibility in selectively and independently positioning the IM nail 200 and/or the bone plate 300 (e.g., surgeons are provided with increased flexibility in positioning the bone plate 300 relative to the IM nail 200). This is, in contrast to known nail-plate combo systems where the bone plate includes threaded screw openings so that the bone screw is fixedly secured to both the bone plate and the IM nail. Prior art nail-plate combos require specific or exact alignment between the threaded openings formed in the bone plate and the IM nail to enable a bone screw to be inserted therethrough. In contrast, in accordance with one or more features of the present disclosure, by providing one or more elongated, non-threaded linking openings 330 in the bone plate 300, and/or one or more locking screw openings 222, and/or one or more locking elongated slots 224 in the IM nail 200, surgeons are provided with increased flexibility to independently position the location of the IM nail 200 and the position of the bone plate 300 while still enabling the surgeon to link the bone plate 300 to the IM nail 200.

In some examples, the range of adjustment provided by the one or more elongated, non-threaded linking openings 330 in the bone plate 300 and/or the one or more locking screw openings 222 or locking screw elongated slots 224 in the IM nail 200, as measured along the axis of the IM nail 200 and/or patient's bone (e.g., along the longitudinal axis of the longitudinal slot of the bone plate and/or IM nail), provides surgeons with the ability to adjust the position of the bone plate 300 relative to the IM nail 200 by 5 mm or more. In some examples, the slot(s) (e.g., the linking openings 330 formed in the bone plate 300 and/or the elongated slot 224 formed in the IM nail 200) may provide surgeons with the ability to adjust the position of the bone plate 300 relative to the IM nail 200 by approximately 15 mm. In other examples, with reference to FIGS. 5D and 5E, the range of adjustment can be defined on one end (FIG. 5D) by inserting a bone screw such as, for example, bone screw 400, through one of the linking openings 330 formed in the bone plate 300 (i.e., linking opening 330 farthest from the end of the bone plate 300) and into the most distal screw opening 220 formed in the IM nail 200) and on the other end (FIG. 5E) by inserting a bone screw such as, for example, bone screw 400, through the other one of the linking openings 330 formed in the bone plate 300 and into the most proximal screw opening 220 formed in the IM nail 200. In such examples, the total range of adjustment can be approximately 55 mm along the axis of the IM nail and bone.

In addition, in accordance with one or more features of the present disclosure, which may be used in combination with, or independently from, the other features disclosed herein, the bone plate 300 may include one or more variable angled nail screw openings 340, which are arranged and configured to receive a larger diameter (“IM”) nail screw 420 as opposed to a more conventional bone screw 402, as will be described in greater detail below.

Thus arranged, in some examples, as illustrated, the bone plate 300 may include one or more elongated, non-locking slots such as, for example, linking openings 330, a plurality of locking (e.g., threaded) screw openings 322, a plurality of variable angled openings 324, and a plurality of variable angled nail screw openings 340. In use, the one or more elongated, non-locking slots (e.g., linking openings 330) may be aligned with one or more elongated slots 224 and/or one or more locking screw openings 222 formed in the IM nail 200 to enable coupling (e.g., linking) thereto. The plurality of locking screw openings 322 and the plurality of variable angled openings 324 enable surgeons increased flexibility to position one or more conventional bone screws 402 through the bone plate 300 while enabling avoidance of the implanted IM nail 200. The plurality of variable angled nail screw openings 340 enabling surgeons increased flexibility to position one or more nail screws 420, as will be described in greater detail below, while enabling avoidance of the implanted IM nail 200.

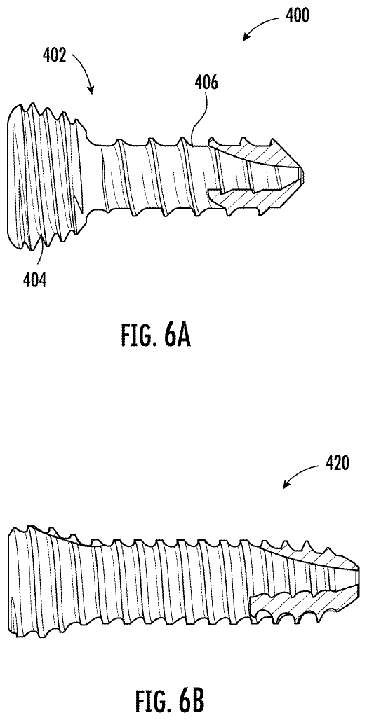

In use, the one or more variable angled nail screw openings 340 are arranged and configured to receive a nail screw 420 (FIG. 6B) optimized for IM nails 200 while still enabling the nail screw 420 to be locked to the bone plate 300 via interaction with the pitch of the threads formed on the nail screw 420. That is, with reference to FIG. 6A, a conventional bone screw 402 is illustrated. In use, the bone screw 402 includes a head portion 404 and a shaft portion 406. As illustrated, the head portion 404 includes threads. Similarly, the shaft portion 406 includes threads. As illustrated, the head portion 404 may include a larger diameter than the shaft portion 406. In addition, the threads formed on the head portion 404 may include a different pitch as compared to the threads formed on the shaft portion 406. As will be appreciated by one of ordinary skill in the art, in use, the threads formed on the shaft portion 406 are optimized for engaging the patient's bone B, while the threads formed on the head portion 404 are optimized for engaging the openings 320 (e.g., locking screw openings 322 or a variable angled openings 324) formed in the bone plate 300.

In contrast, with reference to FIG. 6B, an example of a nail screw 420 in accordance with one or more features of the present disclosure is illustrated. As illustrated, the nail screw 420 may include a continuous thread extending the entire length thereof, or at least a substantially distance thereof (e.g., 85-99 percent). As such, the nail screw 420 may be characterized as being a headless screw. In use, the entire thread, or at least a substantial distance thereof (e.g., 85-99 percent), formed on the nail screw 420 is optimized for implantation into the patient's bone B. For example, the nail screw 420 may be arranged and configured with a larger diameter as compared to conventional bone screws 402. For example, the nail screw 420 may have a diameter of between 4.5 mm and 7.0 mm. In addition, as illustrated, the pitch of the threads of the nail screw 420 is increased as compared to the pitch of the threads used on a conventional bone screws 402 (i.e., the pitch of the threads on the nail screw 420 that interact with the fins is greater than the pitch of the threads formed on the head portion of a conventional bone screw used to interface with metal (e.g., threads, fins, etc.) of the orthopedic implant (e.g., bone plate)). For example, the pitch of the threads formed on the nail screw 420 may be between 2 mm and 3.2 mm, which is in contrast to the pitch of the locking threads on a conventional bone screw, which may be between 0.5 mm and 1 mm. As such, in use, the one or more variable angled nail screw openings 340 are arranged and configured to receive, lock, etc. a nail screw 420 optimized for insertion into a patient's bone B.

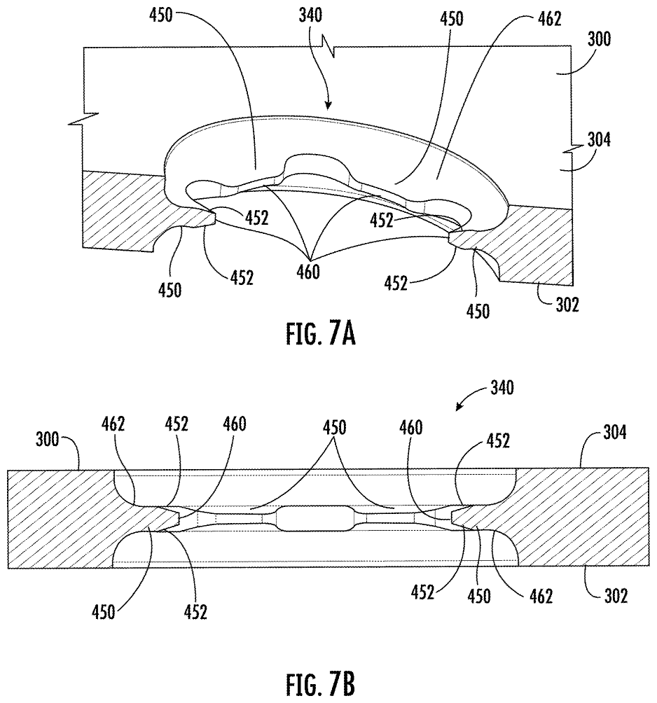

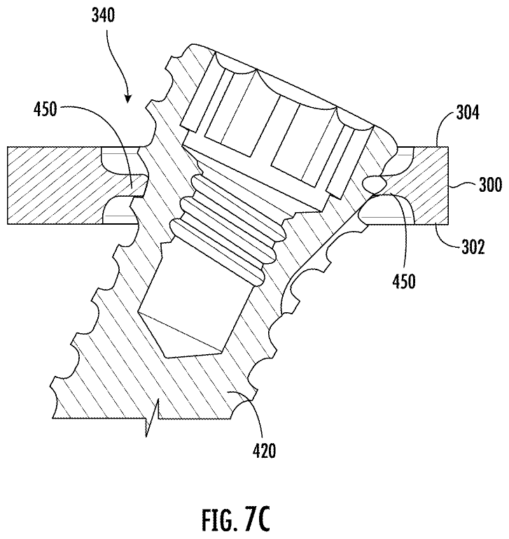

With reference to FIGS. 7A-7C, in order to engage the nail screw 420, which has threads optimized for insertion into a patient's bone and which has a smaller diameter at the proximal end (i.e., head end) thereof in comparison to conventional bone screws 402 (i.e., the proximal or head end of the nail screw 420 has a smaller diameter than the head portion 404 of a conventional bone screw 402), the fins 450 formed in the one or more variable angled nail screw openings 340 include one or more tapers 452. For example, the fin 450 may include a tip 460. In use, the tip 460 may include a thickness of approximately 8/1000 of an inch, which is arranged and configured to deform. As the fin 450 moves inwardly from the tip 460, the fins include a taper 452 to increase the thickness of the fins 450 to the approximate pitch of the threads formed on the nail screw 420. As illustrated, in some examples, the taper 452 may be configured as a dual taper including a taper along the top and bottom surfaces thereof, although this is but one configuration and the fin may include a single taper along the top surface or the bottom surface only. Thus arranged, the fins 450 are arranged and configured to deform to facilitate interdigitation or engagement with the threads formed on the nail screw 420 while still enabling the nail screw 420 to pass therethrough at an angle and into the patient's bone B. That is, the tapered fins 450 include a variable thickness extending from the tip 460 to a base 462 thereof. The thickness at the tip 460 of the fin 450 is arranged and configured to deform to engage the threads formed on the nail screw 420. The thickness tapers to a thickness sufficient to prevent deformation so that the nail screw 420 is prevented from passing therethrough (e.g., fins 450 taper from a deformable tip 460 to a non-deformable thickness adjacent the base 462).

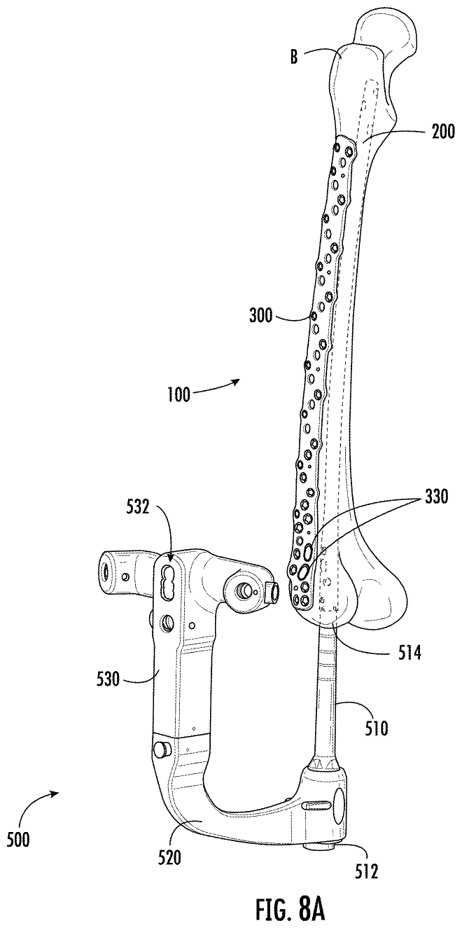

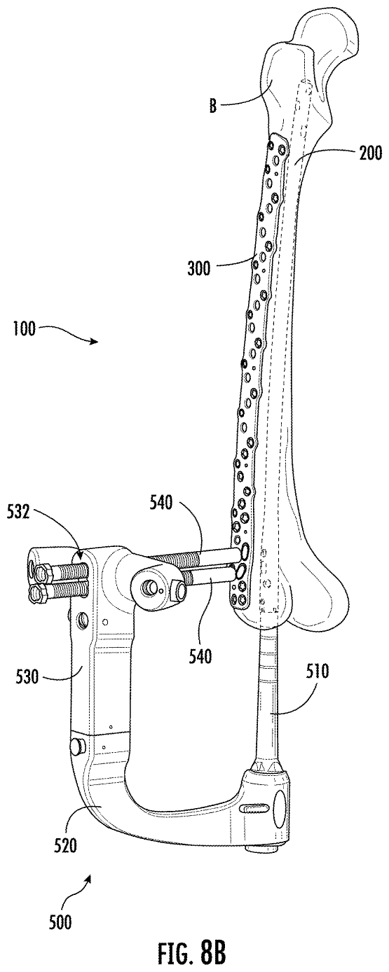

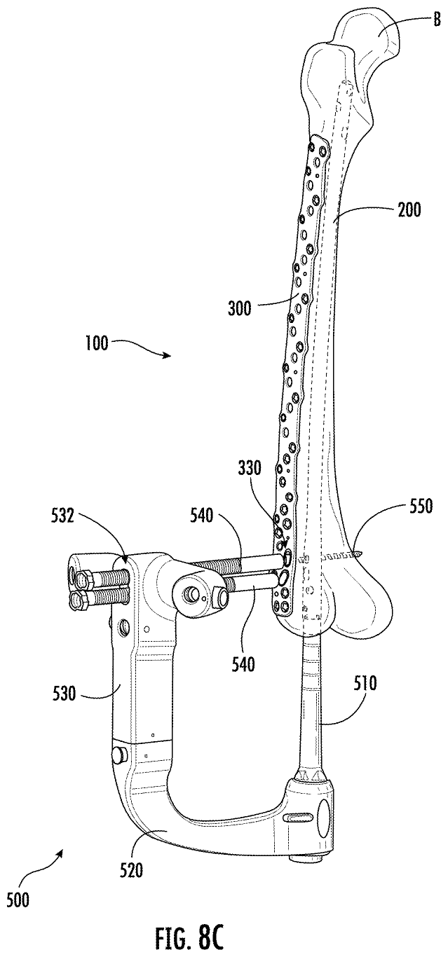

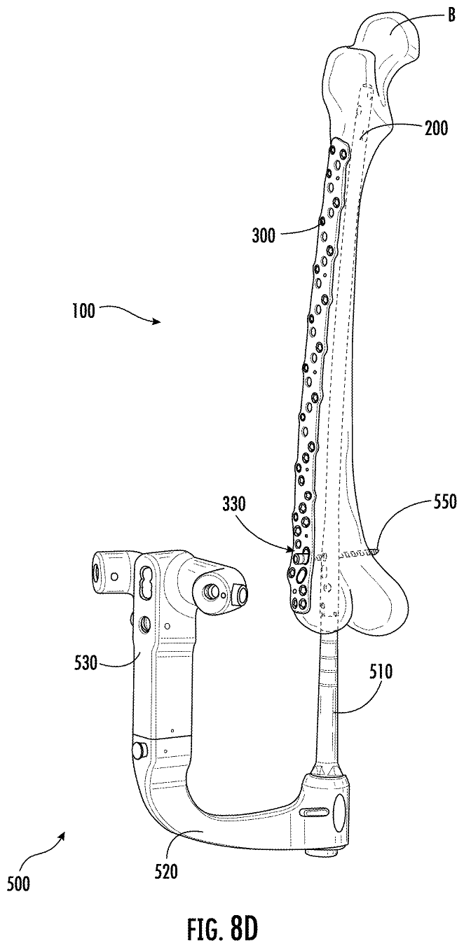

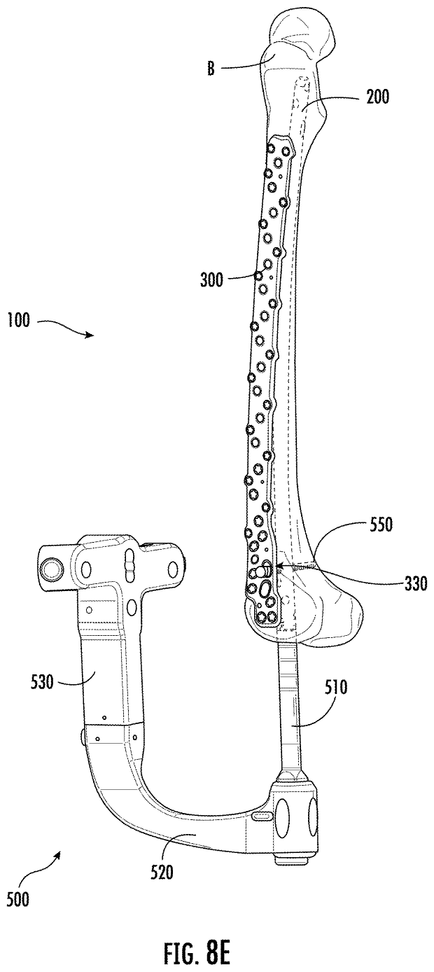

With reference to FIGS. 8A-8E, in accordance with one or more features of the present disclosure, which may be used in combination with, or separately and independently from, the other features disclosed herein, improved instrumentation and corresponding methods of implanting the IM nail-bone plate system 100 is disclosed.

In use, with the IM nail 200 implanted within the intramedullary canal of the patient's bone B and with the bone plate 300 positioned against the patient's bone B, the one or more linking openings 330 formed in the bone plate 300, and the one or more locking screw openings 222 and/or one or more elongated slots 224 formed in the IM nail 200 may be targeted (e.g., one or more of the linking openings 330 formed in the bone plate 300 can be aligned with one or more of the locking screw openings 222 and/or one or more of the elongated slots 224 formed in the IM nail 200 so that a bone screw 400 and/or provisional fixation pin 550, as will be described in greater detail below, can be inserted through the linking opening 330 formed in the bone plate 300 and the locking screw opening 222 or the elongated slot 224 formed in the IM nail 200).

With reference to FIGS. 8A-8E, instrumentation such as, for example, a targeting jig assembly 500, for targeting the one or more linking openings 330 formed in the bone plate 300 and the one or more locking screw openings 222 and/or one or more elongated slots 224 formed in the IM nail 200 is illustrated. As illustrated, the targeting jig assembly 500 includes a guide handle 520, which during use, as will be appreciated by one of ordinary skill in the art, may be coupled to the implanted IM nail 200. For example, as illustrated, the targeting jig assembly 500 may include a rod 510, which, in use, may be coupled to the implanted IM nail 200 (e.g., the rod 510, and hence the targeting jig assembly 500, may be coupled to the first or proximal end portion 212 of the body 210 of the IM nail 200).

In accordance with one or more features of the present disclosure, in use, the guide handle 520 is arranged and configured to swivel relative to the implanted IM nail 200. That is, in use, the guide handle 520 may be arranged and configured to rotationally rotate about a longitudinal axis of the implanted IM nail 200. For example, as illustrated, the guide handle 520 may be coupled to the rod 510. For example, the rod 510 may include a first end 512 and a second end 514. In use, the second end 514 may be coupled to the implanted IM nail 200. The guide handle 520 may be coupled to the first end 512 of the rod 510 such that the guide handle 520 can swivel relative to the rod 510 coupled thereto. Thus arranged, the guide handle 520 can be moved between various positions.

As illustrated, the targeting jig assembly 500 may also include a drop or targeter 530 coupled to the guide handle 520. In use, the drop or targeter 530 includes one or more openings 532 formed therein for targeting the one or more linking openings 330 formed in the bone plate 300 and the one or more locking screw openings 222 and/or one or more elongated slots 224 formed in the IM nail 200. In use, one or more guide sleeves 540 may be inserted into the one or more openings 532 formed in the drop or targeter 530 for targeting the one or more linking openings 330 formed in the bone plate 300 and the one or more locking screw openings 222 and/or one or more elongated slots 224 formed in the IM nail 200. In use, with the one or more linking openings 330 formed in the bone plate 300 and the one or more locking screw openings 222 and/or one or more elongated slots 224 formed in the IM nail 200 targeted, one or more provisional fixation pins 550 may be inserted through the guide sleeves 540 and into the one or more linking openings 330 formed in the bone plate 300 and the one or more locking screw openings 222 and/or one or more elongated slots 224 formed in the IM nail 200. Thus arranged, the bone plate 300 and the IM nail 200 may be provisionally coupled to each other via the provisional fixation pins 550 while enabling the surgeon to still adjust the position of the bone plate 300 and/or IM nail 200 (i.e., the bone plate 300 and the IM nail 200 can be provisionally coupled to each other while still enabling the final position of the IM nail 200 and/or bone plate 300 to be adjusted).

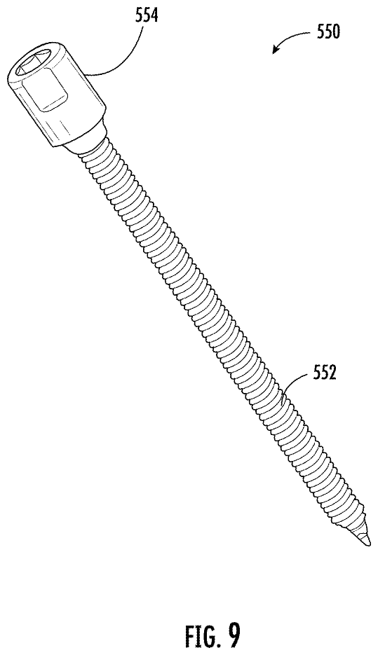

With reference to FIG. 9, in some examples, the provisional fixation pins 550 may include a shaft portion 552 and a head portion 554. In use, the head portion 554 may include a coupling mechanism for receiving, for example, a screwdriver, etc. The shaft portion 552 may include a diameter that substantially corresponds with the minor diameter of the bone screws. Thus arranged, in use, the provisional fixation pins 550 may be inserted through the guide sleeves 540 and into the one or more linking openings 330 formed in the bone plate 300 and the one or more locking screw openings 222 and/or one or more elongated slots 224 formed in the IM nail 200 to link or couple the bone plate 300 to the IM nail 200. However, with the provisional fixation pins 550 inserted, the bone plate 300 and IM nail 200 are still loose so that the final position of the bone plate 300 and/or IM nail 200 can be adjusted. The provisional fixation pins 550 do not threadably engage with the one or more linking openings 330 formed in the bone plate 300 or the one or more locking screw openings 222 or the one or more elongated slots 224 formed in the IM nail 200. In addition, with the inclusion of the elongated non-threaded linking opening 330 formed in the bone plate 300 and/or with the inclusion of the locking elongated slot 224 formed in the IM nail 200, the surgeon can still separately and independently move the bone plate 300 and/or IM nail 200 relative to each other with the provisional fixation pin 550 maintaining a provisional coupling therebetween.

Thereafter, with the provisional fixation pins 550 coupled to the bone plate 300 and the IM nail 200, the guide handle 520, and hence the drop or targeter 530, can be swiveled or rotated relative to the rod 510 thereby enabling the drop or targeter 530 to moved out of the way to provide additional space for the surgeon during the surgical procedure such as, for example, access space for a C-arm, provisional clamps, inserting additional bone screws through the bone plate, etc.

In some examples, the guide handle 520 may be configured to swivel by any suitable mechanism now known or hereafter developed. In some examples, the guide handle 520 may include separate and discrete positions. For example, the guide handle 520 may include a first position for targeting the one or more linking openings 330 formed in the bone plate 300 and the one or more locking screw openings 222 and/or one or more elongated slots 224 formed in the IM nail 200 (FIGS. 1 and 8A-8D). Thereafter, the guide handle 520 may be configured to swivel ±90-degrees (FIG. 8E), although this is but one configuration and the guide handle 520 may include more or less discrete positions or may be continuously rotatable.

In use, with the bone plate 300 provisional coupled to the IM nail 200 via the provisional fixation pins 550, the surgeon can remove the guide sleeves 540 and swivel the drop or targeter 530 out of the way and proceed to couple the bone plate 300 to the patient's bone B using the remaining screw openings (e.g., insert bone screws through the bone plate 300 into the patient's bone B while avoiding the implanted IM nail 200).

In use, with the bone plate 300 coupled to the patient's bone B, the drop or targeter 530 can be returned to its first position, the provisional fixation pins 550 can be removed and one or more screws can be inserted into the one or more linking openings 330 formed in the bone plate 300 and the one or more locking screw openings 222 and/or one or more elongated slots 224 formed in the IM nail 200 using the guide sleeves 540.

Thereafter, as needed, or desired, the guide handle 520, and hence the drop or targeter 530, can be swiveled out of the way and any remaining screws can be inserted into through the bone plate 300 and into the patient's bone B.

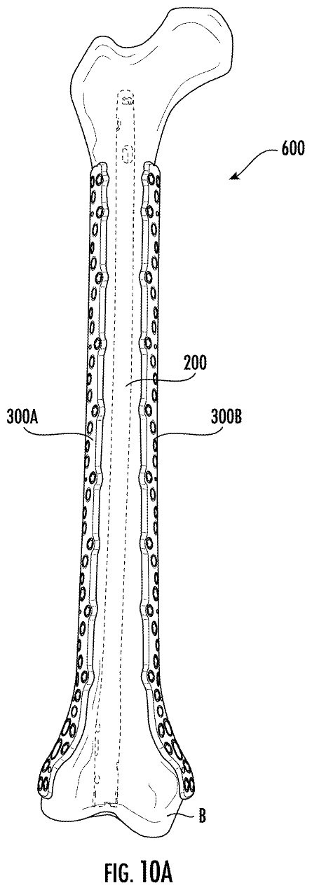

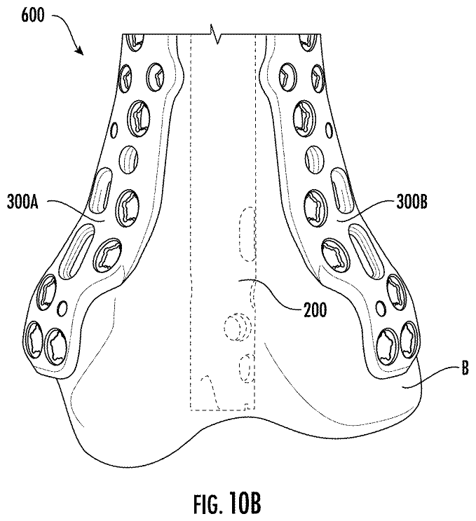

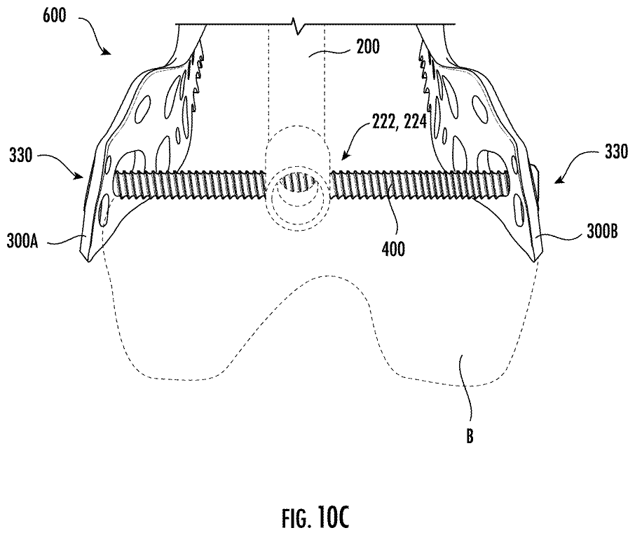

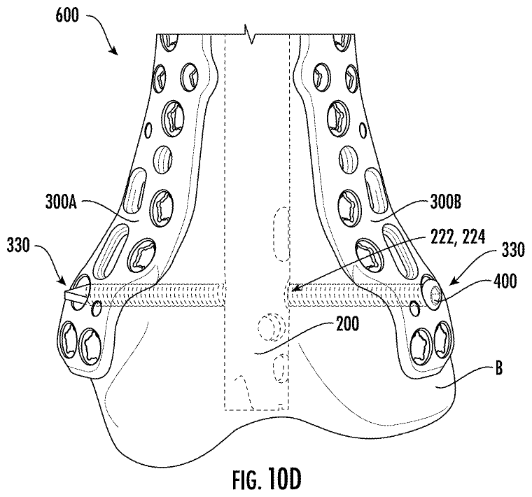

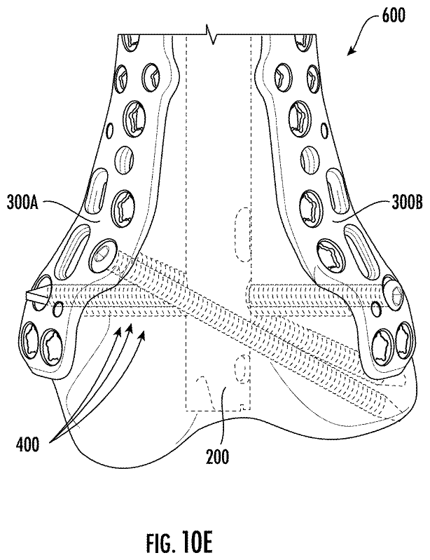

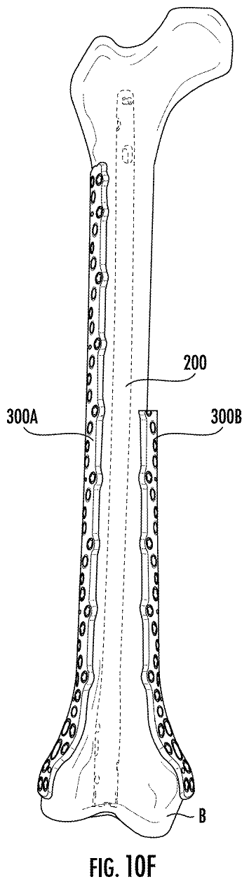

While features of the present disclosure have been illustrated and described in connection with coupling a single bone plate to an IM nail, it should be appreciated that multiple bone plates may be utilized. For example, with reference to FIGS. 10A-10D, an IM nail-bone plate system 600 incorporating an IM nail such as, for example, IM nail 200, and first and second bone plates such as, for example, first and second bone plates 300A, 300B may be utilized. As illustrated, in some examples, the first and second bone plates 300A, 300B may be positioned on either side of the IM nail 200. For example, a first bone plate 300A may be positioned on the lateral side of the IM nail 200 and a second bone plate 300B may be positioned on a medial side of the IM nail 200. Thus arranged, in use, a bone screw such as, for example, bone screw 400, may be positioned such that it passes through the first bone plate 300A through the IM nail 200 and into the second bone plate 300B or vice-versa. For example, the bone screw 400 may pass through a linking opening 330 formed in the first bone plate 300A through one of the locking screw openings 222 or one of the locking elongated slots 224 formed in the IM nail 200 and into, for example, a linking opening 330 formed in the second bone plate 300B or vice-versa. In some embodiments, by extending the bone screw 400 between the first and second bone plates 300A, 300B and/or through the IM nail 200, the bone screw 400 compresses the first and second bone plates 300A, 300B, and hence the patient's bones (e.g., condyles), together.

However, other configurations are envisioned. For example, with reference to FIG. 10E, the bone screw 400 may be configured to pass through one of the first and second bone plates 300A, 300B only while avoiding the other bone plate 300A, 300B and/or IM nail 200. Alternatively, the bone screw 400 may pass through a single bone plate and into the IM nail 200 as previously described, while avoiding the other bone plate. In some embodiments, other bone screws can be utilized to secure the other bone plate.

In some embodiments, the first and second bone plates 300A, 300B may be similarly configured (i.e., the first and second bone plates may have the same length, configuration, etc.). Alternatively, with reference to FIG. 10F, in some embodiments, the second bone plate may be different than the first bone plate. For example, the first and second bone plates 300A, 300B may have different lengths, configurations, etc.

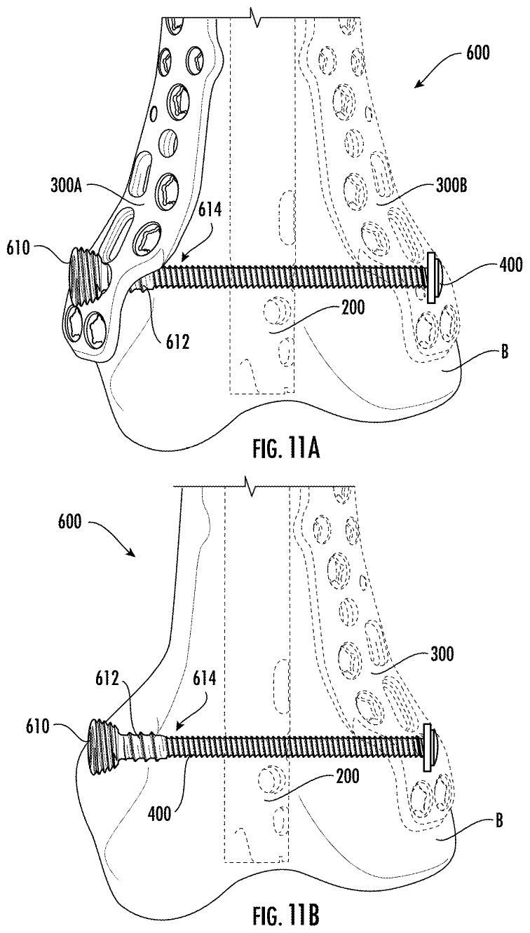

With reference to FIG. 11A, in some embodiments, the IM nail-bone plate system 600 may further include a set screw 610. The set screw 610 may include external threads 612 for engaging the bone plate and/or patient's bone. In addition, the set screw 610 may include an internal bore 614. The internal bore 614 may include threads for threadably engaging the external threads of the bone screw 400. Thus arranged, the set screw 610 can be utilized to threadably engage the bone screw 400 to compress the first and second bone plates 300A, 300B, and hence the patient's bones (e.g., condyles), together. Alternatively, as illustrated in FIG. 11B, the set screw 610 may be utilized as a standalone device to engage the end of the bone screw 400.

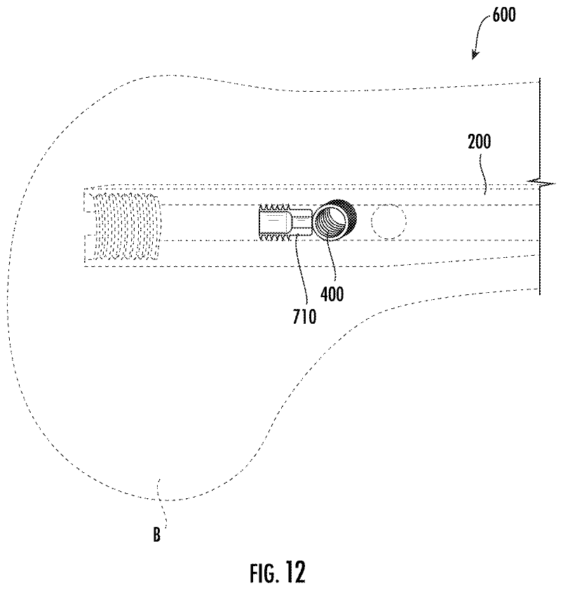

With reference to FIG. 12, in another example, the IM nail-bone plate system 600 may further include an internal set screw 710. The set screw 710 may be positioned within the internal bore of the IM nail 200. The set screw 710 may include external threads for threadably engaging the internal bore of the IM nail 200. In use, the set screw 710 contacts the bone screw 400 passing through the IM nail 200 to prevent backout of the bone screw 400.

As described herein, the one or more variable angled nail screw openings 340 may be provided in a bone plate 300, however, it is envisioned that the variable angled nail screw openings 340 are not so limited and may be used in other orthopedic implants including, for example, acetabular implants, shoulder implants, etc.

The orthopedic implants including, for example, the IM nail, the bone plate, the bone screws, the nail screws, etc. may be manufactured from any suitable material now known or hereafter developed, including, for example, metals, polymers, plastics, ceramics, resorbable, non-resorbable, composite materials, etc. Suitable materials may include, for example, titanium, stainless steel, cobalt chrome, polyetheretherketone (PEEK), polyethylene, ultra-high molecular weight polyethylene (UHMWPE), resorbable polylactic acid (PLA), polyglycolic acid (PGA), combinations or alloys of such materials or any other appropriate material that has sufficient strength to be secured to and hold bone, while also having sufficient biocompatibility to be implanted into a patient's body. In some examples, the bone screws may be manufactured from the same material as the implant. In other examples, the bone screws may be manufactured from a different material as compared to the implant.

The bone screws can be any type of fastener now known or hereafter developed. For example, the bone screws may include any type of external thread including standard or non-standard threads. For example, the external threads can be arranged as a continuous ridge or a non-continuous ridge. The external threads can form a portion of a revolution, one complete revolution, multiple revolutions, a single lead, multiple leads, or any other threads known in the art. Additionally, and/or alternatively, in the case of locking screws, the head portion of the bone screw can include any surface that will engage with and seat within the opening. For example, the head portion can include threads. Alternatively, the head portion can include a series of dimples, ridges, bumps, textured areas, or any other surface that can secure the bone screw.

The bone screw may be any typical bone screw, made out of any appropriate material. The bone screws may include a bore for receiving a driver in order to drive the bone screw through the bone plate and into the patient's bone. The bore may be any size and shape, for example, it may have a hexagonal configuration to receive a corresponding hexagonal driver, a Phillips screw head, a flat-head, a star configuration, Torx, or any other appropriate configuration that can cooperate with a driver to drive the bone screw through the bone plate and into the patient's bone.

The shaft of the bone screws may be fully threaded, partially threaded, or a helical blade, and/or may include one or more tacks, deployable talons, expandable elements, or any feature that allows the shaft to engage the patient's bone. The end of the shaft may be a self-tapping or self-drilling tip.

The foregoing description has broad application. Accordingly, the discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The term “a” or “an” entity, as used herein, refers to one or more of that entity. As such, the terms “a” (or “an”), “one or more” and “at least one” can be used interchangeably herein. The use of “including,” “comprising,” or “having” and variations thereof herein is meant to encompass the items listed thereafter and equivalents thereof as well as additional items. Accordingly, the terms “including,” “comprising,” or “having” and variations thereof are open-ended expressions and can be used interchangeably herein. The phrases “at least one”, “one or more”, and “and/or”, as used herein, are open-ended expressions that are both conjunctive and disjunctive in operation.

All directional references (e.g., proximal, distal, upper, underside, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order, and relative sizes reflected in the drawings attached hereto may vary.

What is claimed is:

1. An intramedullary (“IM”) nail-bone plate system comprising:
an IM nail arranged and configured to be inserted into an intramedullary canal of a patient's bone, the IM nail including a plurality of locking screw openings;
a bone plate arranged and configured to be positioned against the patient's bone, the bone plate including a plurality of screw openings and one or more elongated, non-locking screw openings; and
one or more bone screws;
wherein at least one bone screw is arranged and configured to be inserted into the one or more elongated, non-locking screw opening formed in the bone plate and into one of the plurality of locking screw openings formed in the IM nail.

2. The IM nail-bone plate system of claim 1, wherein at least one of the plurality of locking screw openings formed in the IM nail is configured as an elongated slot so that the at least one bone screw is configured to be inserted through one of the elongated, non-locking screw openings formed in the bone plate and into the elongated slot formed in the IM nail.

3. The IM nail-bone plate system of claim 2, wherein the elongated slot formed in the IM nail and the elongated, non-locking screw openings formed in the bone plate enable independent positioning of the IM nail, the bone plate, or a combination thereof.

4. The IM nail-bone plate system of claim 1, wherein the plurality of screw openings formed in the bone plate include a plurality of locking screw openings and a plurality of non-locking variable angled screw openings.

5. The IM nail-bone plate system of claim 4, wherein the plurality of screw openings formed in the bone plate further include one or more variable angled nail screw openings arranged and configured to receive a larger diameter nail screw.

6. The IM nail-bone plate system of claim 5, wherein the one or more variable angled nail screw openings include a plurality of concavely indented, inwardly protruding fins defining a non-threaded opening for cooperating with threads formed on a nail screw;
wherein each of the fins include a terminal tip, a base, and a tapered surface such that the tip includes a first thickness at the tip arranged and configured to deform, the base includes a second thickness greater than the first thickness, the second thickness sufficient to prevent deformation.

7. The IM nail-bone plate system of claim 6, wherein the nail screw includes threads having a pitch configured to be inserted into a patient's bone.

8. The IM nail-bone plate system of claim 7, wherein the nail screw includes a continuous thread extending an entire length thereof.

9. The IM nail-bone plate system of claim 6, wherein the first thickness is approximately 8/1000 of an inch.

10. The IM nail-bone plate system of claim 9, wherein the second thickness is equal to approximately a pitch of the threads formed on the nail screw.

11. The IM nail-bone plate system of claim 1, further comprising a second bone plate, wherein the at least one bone screw inserted into the one or more elongated, non-locking screw opening formed in the bone plate and into one of the plurality of locking screw openings formed in the IM nail passes through an opening formed in the second bone plate.

12. The IM nail-bone plate system of claim 11, further comprising a set screw arranged and configured to engage a tip of the at least one bone screw inserted into the one or more elongated, non-locking screw opening formed in the bone plate and into one of the plurality of locking screw openings formed in the IM nail and which passes through the opening formed in the second bone plate.

13. The IM nail-bone plate system of claim 11, further comprising an internal set screw positioned within the IM nail, the internal set screw arranged and configured to contact the at least one bone screw inserted into the one or more elongated, non-locking screw opening formed in the bone plate and into one of the plurality of locking screw openings formed in the IM nail and which passes through the opening formed in the second bone plate.

14. A method of repairing a fractured bone, the method comprising:
inserting an intramedullary (“IM”) nail into an intramedullary canal of the fractured bone, the IM nail including one or more locking openings;
positioning a bone plate against the fractured bone B, the bone plate including one or more elongated, non-threaded linking openings;
coupling a targeting jig assembly to the IM nail, the targeting jig assembly including a targeter including one or more openings formed therein for targeting the one or more elongated, non-threaded linking openings formed in the bone plate;
inserting one or more guide sleeves into the one or more openings formed in the targeter;
inserting one or more provisional fixation pins through the one or more guide sleeves, through the one or more elongated, non-threaded linking openings formed in the bone plate, and into the or more locking openings formed in the IM nail; and
optionally, adjusting a position of the bone plate relative to the IM nail.

15. The method of claim 14, further comprising:
removing the one or more guide sleeves; and
rotating the targeter relative to the IM nail to provide increased access space to the surgeon.

16. The method of claim 15, further comprising:
inserting one or more bone screw through one or more openings formed in the bone plate, the one or more bone screws avoiding the IM nail.

17. The method of claim 16, further comprising:
rotating the targeter relative to the IM nail to its original position;
re-inserting the one or more guide sleeves;
removing the one or more provisional fixation pins; and
inserting one or more bone screw through the one or more guide sleeves, through the one or more elongated, non-threaded linking openings formed in the bone plate, and into the or more locking openings formed in the IM nail.

18. The method of claim 14, wherein the targeting jig assembly further comprises:
a rod coupled to the IM nail; and
a guide handle coupled to the rod and the targeter;
wherein the guide handle is rotatably coupled to the rod between a first position, wherein the one or more openings formed in the targeter are aligned with the one or more elongated, non-threaded linking openings formed in the bone plate, and a second position, wherein the one or more openings formed in the targeter are not aligned with the one or more elongated, non-threaded linking openings formed in the bone plate.

19. An orthopedic implant comprising:
a first surface, a second surface, and a plurality of variable angled openings extending from the first surface to the second surface, each of the plurality of variable angled opening including a plurality of concavely indented, inwardly protruding fins defining a non-threaded opening for cooperating with threads formed on a screw;
wherein each of the fins include a terminal tip, a base, and a tapered surface such that the tip includes a first thickness at the tip arranged and configured to deform, the base includes a second thickness greater than the first thickness, the second thickness sufficient to prevent deformation.

20. The orthopedic implant of claim 19, wherein each of the plurality of variable angled opening is arranged and configured to receive a nail screw including threads having a pitch configured to be inserted into a patient's bone.
